(19) United States
(12) Patent Application Publication
(10) Pub. No.: US 2005/0209694 A1
(43) Pub. Date: Sep. 22, 2005

(54) Artificial spinal joints and method of use

(76) Inventor: Marvin P. Loeb, Huntington Beach, CA (US)

Correspondence Address: OLSON & HIERL, LTD., 20 NORTH WACKER DRIVE, 36TH FLOOR, CHICAGO, IL, 60606, US

(21) Appl. No.: 11/076,148

(22) Filed: Mar. 9, 2005

Related U.S. Application Data
(60) Provisional application No. 60/552,619, filed on Mar. 12, 2004.

(51) Int. Cl. A61F 2/44

(52) U.S. Cl. 623/17.11; 606/61

(57) ABSTRACT

An artificial spinal joint, consisting of a flexible or rigid member or a pair of moveably-joined, flexible or rigid segments, is formed into a spring-like shape, whose distal ends have feet with slots through which screws can be inserted to attach the artificial joint to vertebra whose facets (joints) are non-functional. The artificial spinal joint is able to prevent subluxation of the spine, while retaining the mobility of the spine and permitting angular deflection of the vertebra above and below a non-functional spinal joint. A jig is used to position tools and make passageways for screws to attach the artificial spinal joint to the vertebra or its pedicles or facets in a minimally invasive procedure. The rigid members or segments are bio-compatible and may be made of titanium, a titanium alloy, tantalum, medical grade stainless steel or carbon fibers in a matrix of a rigid, durable plastic. The flexible members or segments may be made of spring steel coated with a durable, bio-compatible material, small diameter carbon fibers in a flexible, durable plastic matrix, or a single shape or dual shape, superelastic memory metal. The feet, made of any of the rigid or flexible materials described above, may also be moveably attached to the proximal ends of the members or segments. Having the feet moveably attached to the segments facilitates insertion of the artificial spinal joint into the body by folding the feet parallel to the axis of the segments during insertion, and then unfolding the feet for attachment to the vertebra or its pedicles or facets. The artificial spinal joint may be inserted and attached to vertebra whose facets are non-functional in minimally invasive, moderately invasive or conventional surgical procedures.

### CROSS-REFERENCE TO RELATED APPLICATION

This application claims benefit of U.S. Provisional Patent Application No. 60/552,619, filed Mar. 12, 2004, which is incorporated herein by reference.

### BACKGROUND OF THE INVENTION

Scoliosis (abnormal curvature of the spine) or spondylisthesis (displacement of one vertebra with respect to another, called subluxation) is often caused by disease, damage or malfunction of the joints that help anchor the vertebra in the musculature of the back and enable the vertebra to tilt with respect to each other as we bend or move. This condition is presently treated, when exercise and braces fail, by one or more expensive, lengthy surgeries. In these procedures, the facets (joints) of the vertebra are removed and a lattice of rigid metal rods or plates, usually titanium, are attached to the vertebra with screws, forcing the spine into a more normal curvature. These procedures immobilize the spine and adversely affect the patients' quality of life.

In other cases, the facets of the spine are no longer functional, due to damage or disease, such as arthritis, and cause significant pain. This condition is treated by attaching the same lattice of rods or plates to the affected vertebra and immobilizing them, as described above.

Another type of spinal disease is degeneration of spinal discs. While degenerated discs can arise from damage or disease, most degeneration of discs is due to dehydration, which causes them to become thinner. This is why persons become shorter as they grow old. As the disc becomes thinner, the vertebra move closer together and pinch the nerves that lie about the exterior of the disc, causing severe, unrelenting pain.

While experimental artificial discs, nucleus replacement devices, and other spacers are being tested in clinical studies, the conventional surgical procedure to treat a degenerated disc is called fusion surgery. This procedure entails a sizeable incision, significant blood loss, the risks of infections and general anesthesia, a 3-7 day hospital stay, significant post-operative pain, a recuperation period of two to four months and a good or excellent success rate, based upon standardized pain scores, of only 40% to 77%, according to studies published in medical journals.

In fusion surgery, some or all of the lamina (bony appendages of the vertebra) are ground-away, the degenerated disc is removed, two metal “cages”, typically made of titanium, are inserted as spacers to maintain the space between the vertebra. The cages and/or the space between the vertebra is packed with autologous (self) bone removed from the patient's hip (which requires another surgical procedure and additional cost) or allograft (cadaver) bone. In about 60% of the cases, the vertebra grow into and “fuse” with the inserted bone, creating a single bony structure. In some cases, cages are not inserted and the entire space between the vertebra is filled with autologous or allograft bone.

While the fusion process is proceeding, which can take months, metal rods or plates are attached with screws, to the vertebra, the pedicles (bony extensions of the vertebra) or the facets (joints) of the vertebra above and below the removed disc, to prevent movement of the vertebra. Recently, the FDA approved the sale of a bone morphogenic protein, a bone growth-stimulating agent, which has been shown to increase the success of the bone fusion procedure, but at substantial additional cost.

If the fusion process is successful, the pain is relieved. However, the fused portion of the spine has been immobilized and, as the body bends, the discs above and below the fused portion of the spine are subject to greater pressures and may become damaged, creating new sources of pain. Many persons require one or more subsequent fusion procedures to treat the discs above and below the fused vertebra, each at a cost of $60,000 or more, until their spine has become largely immobile and their quality of life has deteriorated.

Even if the disc is not sufficiently degenerated to require the insertion of an artificial spinal disc, nucleus replacement or other spacer, if the facets are non-functional, the lattice of rods or plates is still needed to keep the vertebra from moving with respect to each other, called subluxation.

The surgical treatment of scoliosis, spondylolisthesis, diseased facets and degenerated spinal discs is a major burden on the American healthcare system. In the United States, approximately 400,000 “fusion” surgeries to treat these conditions are performed each year at a cost of about $60,000 per case or $24 billion annually, not including physical therapy and rehabilitation costs, the two to six months the patients are out of work and other indirect costs.

It would be desirable to create a solution to the problems of scoliosis, spondylolisthesis, painful spinal joints and degenerated spinal discs and eliminate the need for surgery and the attachment of the lattice of rods or plates, while enabling the spine to retain some or all of its mobility.

### SUMMARY OF THE INVENTION

The objective of the invention described herein is to provide artificial spinal joints to replace one or more of a patient's non-functional facets in either a minimally invasive procedure or in moderately invasive or conventional surgical procedures.

Recently, new spinal intervertebral supports or spacers, have been developed for treating a degenerated disc in persons with intact (functional) facets, bony extensions of the vertebra that function as joints and allow the vertebra to tilt with respect to each other. Some spacers are artificial devices that replace the nucleus of the disc or the entire disc, or are simply intervertebral, height-maintaining devices.

Some spacers are made of a metal, such as titanium or tantalum, a plastic, such as high density polyethylene or polyethyl ether ketone, a combination of metal and plastic components or sacks containing a hydrogel (a material that swells in volume as it absorbs water). Insertion of such spacers typically requires the vertebra to be spread apart with a distraction tool, like an automobile jack, which requires a substantial incision. Such spacers are often referred to as dynamic stabilization devices, as they maintain some or all of the spine's mobility.

A spacer which atraumatically spreads the vertebra apart, without the need for a spreader or jack, is the Spinal Stabilization Device described in co-owned patent application number PCT/US03/19150, which is fully incorporated herein by reference. The aforementioned Spinal Stabilization Device is inserted posterolaterally in a minimally invasive, outpatient, endoscopic procedure to treat a degenerated lumbar (lower back) disc through a 6-8 mm (¼ to 1/3 inch) puncture that is expanded to about 10-12 mm (0.4 to 0.5 inches) in diameter.

Such a device can also be used to treat a degenerated lumbar disc in a moderately invasive surgical procedure with a one inch or larger opening, sometimes referred to as a Micro Endoscopic Discectomy procedure. The delivery cannula in such procedures can have an outside diameter of 25 mm (1 inch) or larger, and its distal end can be further expanded to provide a larger working area. However, such procedures require hospitalization, albeit shorter in duration than conventional fusion surgery. If such a device is used in a conventional surgical procedure to treat degenerated lumbar discs, with an incision of about 4 inches or larger, a delivery cannula may not be needed. Spacers with smaller diameters would be used to treat degenerated thoracic (upper back) or cervical (neck) discs, with commensurately smaller delivery cannulas, as these discs decline in size in ascending order.

While the Spinal Stabilization Device described above and other spacers can maintain the proper intervertebral space and provide a single pivot point between the vertebra, the patient's facets (joints) must be functional or subluxation can result. In severely degenerated “or collapsed” discs, the facets are usually diseased, often due to arthritis, and often are no longer functional.

As a result, in addition to inserting a spacer to maintain the proper intervertebral space, it would be desirable to be able to attach an artificial joint to the vertebra above and below the non-functional facets, preventing subluxation and enabling the vertebra to tilt with respect to each other, providing an effect similar to that of the spine's normal joints.

In patients with scoliosis, spondylolisthesis, or painful, diseased or damaged facets, where the disc is not sufficiently degenerated to require the insertion of a spacer, a flexible artificial spinal joint of the present invention can be inserted, attached and function in the same manner as the facets.

For use in lumbar or thoracic vertebra, a flexible artificial spinal joint includes a flexible rod with flattened feet at its ends. For use in cervical vertebra, a flexible artificial spinal joint is provided with a flexible plate, comprising a flexible body or bridge between two flattened feet. The rod or the bridge of the plate is formed into a spring-like shape, with one or more circular apertures or a slot for screws in each flattened feet. While either circular apertures or a slot may be used, all references to slots herein shall be understood to include circular apertures as well. In addition to flexibility enabling the rod or plate to function as a joint, its flexibility enables the body of the rod or the bridge of the plate to be compressed and the feet to be bent to permit the device to fit within a cannula for delivery to the vertebra to which it is to be attached.

Two artificial spinal joints are usually attached to the lumbar (lower back) or thoracic (upper back) vertebra, typically spaced apart, like the facets (joints) of the vertebra, for example, at about 4 to 5 o'clock and 7 to 8 o'clock, with 6 o'clock being the patient's posterior. In the case of cervical (neck) vertebra, usually one flexible plate is used at about 10 to 2 o'clock, as cervical vertebra are smaller in size than thoracic or lumbar discs.

Artificial discs, the Spinal Stabilization device described above and other intervertebral spacers must be able to withstand pressures of about 700 lbs. per square inch or more over a period of many years. However, an artificial spinal joint (functioning as a facet) need only bear about 20% to 30% of the pressures which such spacers must withstand. Still, they must be sufficiently durable to function over a period of 50 years or more.

A flexible rod or plate can be made of spring steel, coated, for example, with a polymer such as phosphorycholine, a ceramic such as magnesia partially stabilized zirconia (MgO—PSZ) which has been treated with a carbon dioxide laser to enhance cell adhesion, titanium powder deposited in a vacuum deposition process, or other strong, flexible and biocompatible material, as known in the art.

Instead of metal, the flexible rods or plate may be made of a carbon fiber composite, such as very small diameter carbon fibers in a matrix of a durable, flexible plastic, such as polyethyl ether ketone, known as PEEK, which is manufactured by companies such as Invibio, Inc. of San Juan Capistrano, Calif., and machined or formed into desired shapes by companies such as Sparta Composites of San Diego, Calif., and others. Any other durable and flexible plastic can be used. The addition of very small diameter carbon fibers to PEEK or other strong, durable, plastic increases the strength of the combination, allowing a thinner or narrower device, with greater flexibility, to be employed.

Preferably, the rod or plate may be made of a superelastic memory metal such as, for example, a suitable nickel-titanium alloy. Such compositions are generally known as a NiTi or Nitinol alloy, and are fabricated, for example, by Memry Corporation of Bethel, Conn., or Shape Memory Applications, Inc. of San Jose, Calif. Such a rod or plate can be heat-treated to form a specific shape and will return to its heat-treated shape, no matter how many times it has been deformed by being straightened-out or bent into another shape.

In addition to flexing, like a spring, such a rod or plate may be compressed, for example, in a minimally invasive procedure to replace a non-functional joint in lumbar vertebra, for insertion through a delivery cannula with an outside diameter of about 10 to 12 mm. For use in a moderately invasive procedure, the delivery cannula can have an outside diameter of 25 mm or larger, prior to its expansion. When the device exits the delivery cannula, the rod resumes its heat-treated shape. If used in a conventional surgical procedure, the 4″ or larger incision permits the flexible artificial spinal joint to be inserted with or without a delivery cannula.

The rod or plate of a flexible, artificial spinal joint can be made into a “V”, “C”, “W” or “loop” spring-like shape, a coiled spring shape or any other desired shape, using the flexible materials, as described above. For use in a thoracic vertebra, the rods and the delivery cannula must be smaller, as thoracic vertebra decline in size in ascending order. For use in cervical vertebra, the plate and the delivery cannula are even smaller, as the size of cervical vertebra also decline in ascending order.

In a preferred embodiment, the artificial spinal joint is moveable, consisting of at least two rod or plate segments moveably joined together about a pivot point, such as a rivet or pin, or formed into a hinge, ball joint or other moveable junction, as known in the art. The segments can be flexible and made of the flexible materials described above, enabling them to be compressed for insertion through a delivery cannula.

Alternatively, the segments can be rigid and made of materials such as titanium, a titanium alloy, tantalum (which has about the same hardness or modulus of elasticity as bone) or other very strong, durable and biocompatible metal, or carbon fibers in a matrix of a strong, durable and biocompatible plastic, as described above. Non-flexible embodiments of such moveable artificial spinal joints can be attached to the vertebra in a conventional surgical procedure, without a delivery cannula, or in a moderately invasive surgical procedure through a delivery cannula with an outside diameter of about 25 mm (1 inch) or larger, provided the size of the artificial spinal joint is sufficiently small to not require the feet to be bent to fit within the delivery cannula.

If a spacer, such as the aforementioned Spinal Stabilization Device, is inserted into a lumbar (lower back) or thoracic (chest) disc from a posterolateral approach, one of the flexible rods or flexible jointed rods described above can be compressed and inserted through the same delivery cannula as the spacer. A similar flexible rod or flexible, jointed rod is compressed and inserted from a posterolateral approach on the patient's other side, through a similar incision and delivery cannula.

If an anterior or anterolateral approach is used for the treatment of cervical (neck) vertebra, usually only a single flexible plate, with feet and slots sufficiently wide to each accommodate one or more screws would be used. If a spacer is inserted from an anterior or anterolateral approach, the plate can be compressed and inserted through the same delivery cannula as the spacer. If a second plate is needed, it would be compressed and inserted through a second delivery cannula, spaced apart from the first, on the patient's contralateral side.

The flexible or moveable artificial spinal joint described above, when attached to the vertebra, functions like a normal spinal joint, enabling the vertebra to tilt with respect to each other at an angle of up to about 20° for lumbar or thoracic vertebra or at greater angles for cervical (neck) vertebra, while preventing subluxation.

In a minimally invasive procedure, an insertion tool made, for example, of titanium, a titanium alloy or medical grade stainless steel, is removably attached to the center of one of the rods or the plate. The insertion tool may have a threaded extension projecting from its distal end. A short metal cylinder, with a central, threaded bore can be welded or otherwise attached to the center of the rod or plate or be formed as a part of the rod or plate, enabling the insertion tool to be threadingly engaged therewith. Alternatively, the threaded bore may be made in the center of the rod or plate. To avoid the weakening the center portion of the rod or plate due to the presence of the through bore therein, the area of the rod or plate about the bore opening can be widened or thickened to maintain its strength and durability.

After a small incision is made and a guidewire or trocar is inserted up to the vertebra, tissue expanders are used to make a passageway up to the vertebra, and the delivery cannula is inserted. The surgeon selects a rod or plate of a size appropriate for attachment to the vertebra, based on the distance between the desired points of attachment. The rod or plate is attached to the insertion tool, compressed, inserted into the delivery cannula and advanced to the distal end of the cannula. While holding the rod or plate in place with the insertion tool, when the delivery cannula is retracted, the rod or plate resumes its original, unconstrained shape, with its flattened feet extended and the slots positioned over the desired points of attachment to the pedicles, facets or vertebra. The insertion tool can be used to adjust the position of the rod or plate, and its position can be confirmed by x-ray imaging, if desired.

Screws, typically made of titanium or other durable, biocompatible material, are removably attached to a driver, e.g., a screwdriver, as known in the art, advanced through the passageway and the slots in the feet of the rod or plate and screwed through the pedicles or facets of the vertebra into the vertebral body, or directly into the vertebral body, itself, as known in the art. While the feet can contain one or more circular apertures, slots in the feet are preferred, as they provide additional positioning flexibility for the screws.

Optionally, the edges of the slots can be scalloped, i.e., in the form of a continuous series of circle segments, preferably about one-fourth to one-half circles, preventing movement of the foot from its position on the vertebra, in the event a screw becomes loose over time. All references to slots herein shall be understood to mean ordinary slots, slots with scalloped edges for fasteners such as screws, and the like.

To enable the fasteners to be inserted through the slots in the flat distal ends of the rod or plate and secured into place, without having to greatly expand the initial incision or make additional large incisions, a positioning fixture or jig is inserted over the insertion tool while it is still attached to the center of the rod or plate. To properly align the jig with the insertion tool, the exterior of the insertion tool can be round with an external ridge or key along its length, and the jig may have a round channel with an inside diameter slightly larger than the outside diameter of the insertion tool, with a depression or keyway slightly larger in size than the ridge or key of the insertion device.

Alternatively, the insertion tool can have the depression or keyway, and the jig can have the ridge or key, or the insertion tool can have a square or rectangular exterior and the jig can have a square or rectangular internal channel which is slightly larger in diameter than the exterior of the insertion tool. Any other shapes that fix the position of one with respect to the other can be employed.

Two arms extend laterally from the jig, with hollow guideways at the distal ends of the arms. The hollow guideways are aligned parallel to the axis of the insertion tool. The inside diameters of the hollow guideways are just slightly larger than the outside diameter of the fasteners, e.g., screws. The arms extend laterally from the jig a desired distance. The surgeon selects a jig with properly sized arms, based on the distance between the slots in the feet of the selected, unconstrained rod or plate. The length of the arms may also be adjustable by means known in the art.

After the flexible rod or plate has been inserted through the delivery cannula, the delivery cannula is retracted and the rod or plate resumes its original, unconstrained shape. Without disengaging the insertion tool from the center of the rod or plate, the jig is inserted over and moved down the insertion tool to the surface of the body. Guidewires or trocars, with a cylindrical upper portion with an outside diameter just slightly smaller than the inside diameter of the hollow guideways, are inserted through the guideways. The lower portions of the guidewires or trocars can be the same diameter as the upper portion, in the case of a trocar, or significantly smaller in diameter, in the case of a guidewire, both of which preferably terminate in sharply pointed distal ends.

The guidewires or trocars are inserted through the hollow guideways attached to the arms and advanced through the back and through the slots in the feet of the rods or plate to the desired attachment location on the vertebra or its pedicles or facets. The guidewires or trocars may be removed, and tissue expanders may be used to widen the passageways for the screws, if desired. The fasteners are each removably attached, as known in the art, to a driver whose body has an outside diameter just slightly smaller than the inside diameter of the hollow guideways. The fasteners are inserted through the hollow guideways, the passageways created by the trocars or guidewires (and tissue expanders, if desired) and the slots in the feet and screwed into place. X-ray guidance can be employed to facilitate the procedure.

In a further preferred embodiment, the rod or plate of a flexible artificial spinal joint, or the rod or plate segments of a moveable artificial spinal joint, can be made of a dual shape, superelastic, memory metal alloy, such as, for example, a suitable nickel-titanium alloy, which can be heat-treated to assume one shape at a temperature below about 27° C. (80° F.), for example, and a different shape at a temperature of above about 27° C. (80° F.) or higher, for example, provided the heat-treated temperature of the second shape is significantly higher than that of the first shape.

For example, a flexible or moveable artificial spinal joint made of such a dual shape NiTi alloy can be selected by the surgeon, based on the distance between the points on the vertebra or its facets or pedicles to which the rod or plate is to be attached. The rod or plate can be cooled below about 0° C., such as in a refrigerator or an ice water bath, for example, at which temperature it has a shape which enables it to be inserted through a delivery cannula, without its having to be manually compressed.

After the rod or plate, attached to the insertion tool, has been advanced through the delivery cannula up to the vertebra, the delivery cannula is retracted. When the rod or plate reaches a temperature of about 27° C. (80° F.) or higher, for example, it reverts to its second, expanded shape, which can be any of the shapes described below, with its feet, each containing a slot for one or more screws, extended the proper distance for attachment to the desired points on the facets, pedicles or vertebra.

This avoids having to forcibly compress the rod or plate to make it insertable through the delivery cannula. Also, if it becomes necessary to remove the rod or plate, after removal of the screws, cold, sterile saline or water can be infused and, when the rod or plate has cooled to below about 27° C. (80° F.) or less, it reverts to its first shape and can be removed through the delivery cannula by attaching and withdrawing the insertion tool.

In one embodiment of a moveable artificial spinal joint, the distal ends of the rod or plate segments are disc shaped with a central cylindrical aperture, enabling them to be attached together with a rivet or pin or the like. A metal flange may be welded to or made an integral part of the disk-shaped distal end of one of the rod or plate segments. The flange extends over the disk-shaped distal end of other rod or plate segment and enables the above described cylinder, with a central, threaded bore opening, to be attached to or formed as a part of the flange, with the cylinder positioned at the center of the junction of the rods or plate segments. Alternatively, the threaded bore opening can be made in the flange, itself, over the junction of the distal ends of the rods. The other ends of the rod or plate segments have flattened feet with slots for fasteners such as screws and the like.

In another embodiment, the distal ends of the rod or plate segments can be formed into a hinge, preferably consisting of three flanges. Two of the flanges are on the distal end of one rod or plate segment, spaced apart, and one flange on the distal end of the other rod or plate segment is disposed in the space between the other two flanges. All three flanges are movably curled about the shaft of a pin or rivet or the like, forming a hinge. A cylinder, with a central, threaded bore opening, as described above, can be welded or otherwise attached to or formed as a part of the middle flange of the hinge. Alternatively, the threaded bore opening can be made in the middle flange of the hinge. The other ends of the rod or plate segments have flattened feet with slots for screws. Any other number of rod or plate segments and flanges may be used.

In a most preferred embodiment, the distal end of one rod or plate segment consists of a ball, and the distal end of the other rod or plate segment consists of a cup, which is slightly larger in diameter and moveably extends over at least about 60% or more of the exterior of the ball, forming a ball joint, as known in the art. The other ends of the rod or plate segments have flattened feet with slots for screws, as described above.

The ball joint enables movement of one rod or plate segment with respect to the other at any angle. The cup has an opening to allow movement within the cup of the rod or plate segment that terminates in the ball. The opening in the cup may be made of a size that limits the degree of deflection of one rod or plate segment with respect to the other and, hence, the angular deflection of the vertebra to which they are attached. The cylindrical shape with a central, threaded bore hole can be attached to or formed as a part of the cup. Alternatively, the threaded bore opening can be made in the distal end of the cup, itself.

In a further embodiment, a rigid metal or flexible plastic sleeve may be positioned about the distal end portions of the rod or plate segments, proximal to the movable pin, hinge or ball joint junction, to prevent excessive angular deflection of the rod or plate segments and, hence, the angle of deflection of the vertebra.

If the vertebra to be treated are thoracic, which decrease in size in ascending order from the lower to the upper thoracic discs, the size of the flexible rods and the dimensions of the delivery cannula are smaller than those for lumbar vertebra. The rods are generally inserted from a posterolateral approach and are attached in the same way and function in the same manner. If the vertebra to be treated are cervical, the plate described above is still smaller, and the dimensions of the delivery cannula are less, as cervical vertebra also decline in size in ascending order, from the lower to the upper cervical discs. The plate is typically inserted from an anterior or anterolateral approach, attached in the same way and functions in the same manner.

### BRIEF DESCRIPTION OF THE DRAWINGS

FIG. 1 is a broken away perspective view of a delivery cannula, in which a device of the present invention is disposed;

FIGS. 2-13 are perspective views of various embodiments of the device of the present invention;

FIG. 14 is a broken cut-away, perspective view of a delivery cannula in which the device of FIG. 13 is disposed;

FIG. 15 is a perspective view of a delivery component of the device of the present invention;

FIG. 16 is a cross-sectional view of the delivery component attached to the device of FIG. 2;

FIG. 17 is a perspective view of the positioning and delivery components and the device of FIG. 2 in use;

FIG. 18 is a perspective view of an alternate embodiment of the device of the present invention;

FIG. 19 is a perspective view of a preferred embodiment of the device of the present invention; and

FIG. 20 is a perspective view of another preferred embodiment of a device of the present invention in use.

### DETAILED DESCRIPTION OF THE INVENTION

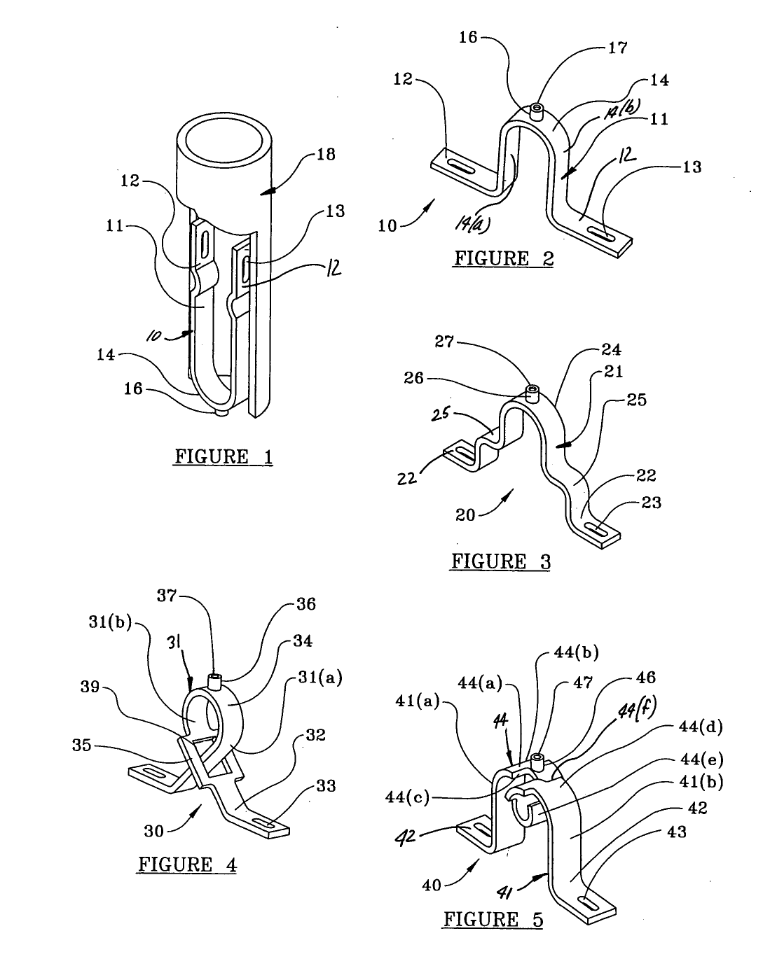

FIG. 1 shows flexible rod device 10, for attachment to lumbar or thoracic vertebra, in its constrained shape within delivery cannula 18. Device 10 consists of a flexible, elongate rod or bar body 11 and feet 12 with slots 13. The center bridge portion 14 of rod body 11 is “U” shaped, as further illustrated in FIG. 2. Cylinder/pin 16 may be formed as a part of or attached to the top surface of “U” shaped center bridge portion 14 and contains a threaded bore aperture 17 (not separately shown) defined and extending therethrough. All of the devices of FIGS. 2-13 and 19-20 are likewise adapted to be constrained or disposed within delivery cannula 18.

As seen in FIG. 2, flexible rod device 10 is shown in its unconstrained shape, prior to being confined within delivery cannula 18 of FIG. 1 or after being ejected therefrom. In its unconstrained shape, center portion 14 defines a flexible, generally U-shaped member having a pair of spaced-apart parallel legs 14(a) and 14(b) and a pair of feet 12 that extend generally normally outwardly from the respective distal ends of the legs 14(a) and 14(b) respectively. Threaded bore aperture 17 in pin 16 enables the threaded distal end portion 132 of insertion tool 130 shown in FIG. 15 to be threadingly and removably secured to the center of flexible rod 10, as shown in FIG. 16. Alternatively, bore opening 17 may be made unitary with the center portion 14 of rod body 11, which may be widened or thickened to maintain its strength and durability.

FIG. 3 illustrates an alternate flexible rod/bar device embodiment 20 in its unconstrained shape. In this embodiment, in addition to flexible “V” shaped center bridge portion or member 24, secondary rounded outwardly protruding shoulders or steps 25 are formed on rod body 21 between the ends of member 24 and feet 22 which define slots 23 allowing movement of feet 22 about the axis of center portion 24 and about the axis of both of the steps 25 when the rod 20 is bent and constrained within the interior of cannula 18. Pin 26 is similar to pin 16 and defines a threaded bore 27.

Another flexible rod/bar device embodiment 30 which functions like a spring is shown in FIG. 4 in its unconstrained shape. In this embodiment, rod body 31 defines a first flexible, elongate rod leg segment 31(b) having a widened lower portion 35 defining a generally rectangularly shaped through slot 39 and a second flexible rod body leg segment 31(a) which loops downwardly and extends through the slot 39 in leg segment 31(b). Rod leg segment 31(b) is widened or thickened to compensate for slot 39 and maintain the strength and durability of rod leg segment 31(b). Feet 32 with slots 33 extend outwardly from the distal ends of the rod leg segments 31(a) and 31(b) respectively. The leg segments 31(a) and 31(b) together define a generally circular center rod member 34. Pin 36 is similar to pin 16 and defines a threaded bore 37 extending upwardly from the top face of member 34.

FIG. 5 shows another flexible bar embodiment 40 in its unconstrained shape. In this embodiment, body 41 includes a flexible center bridge portion 44 defining a first rod body leg segment 41(a) having an end portion 44(a) with a widened shoulder 44(b) and cut-out 44(c) for maintaining the width, strength and durability of body leg segment 41(a). Center bridge portion 44 also defines a body leg segment 41(b) having an end portion 44(d) defining a widened shoulder 44(e) and cut-out 44(f) also to maintain the width, strength and durability of body leg segment 41(b). Cylinder/pin 46 with threaded bore 47 can be formed as part of or attached to the top of the portion 44(a) of body leg segment 41(a), as shown, or alternatively to the top of the end portion 44(d) of body leg segment 41(b). Cut-outs 44(c) and (f) also serve to limit the extent to which flexible bar 40 can be expanded or compressed and, hence, limits the angular deflection of one vertebra with respect to the other. Feet 42 extend generally unitarily perpendicularly outwardly from the respective ends of leg segments 41(a) and 41(b) opposite the end portions 44(a) and 44(d).

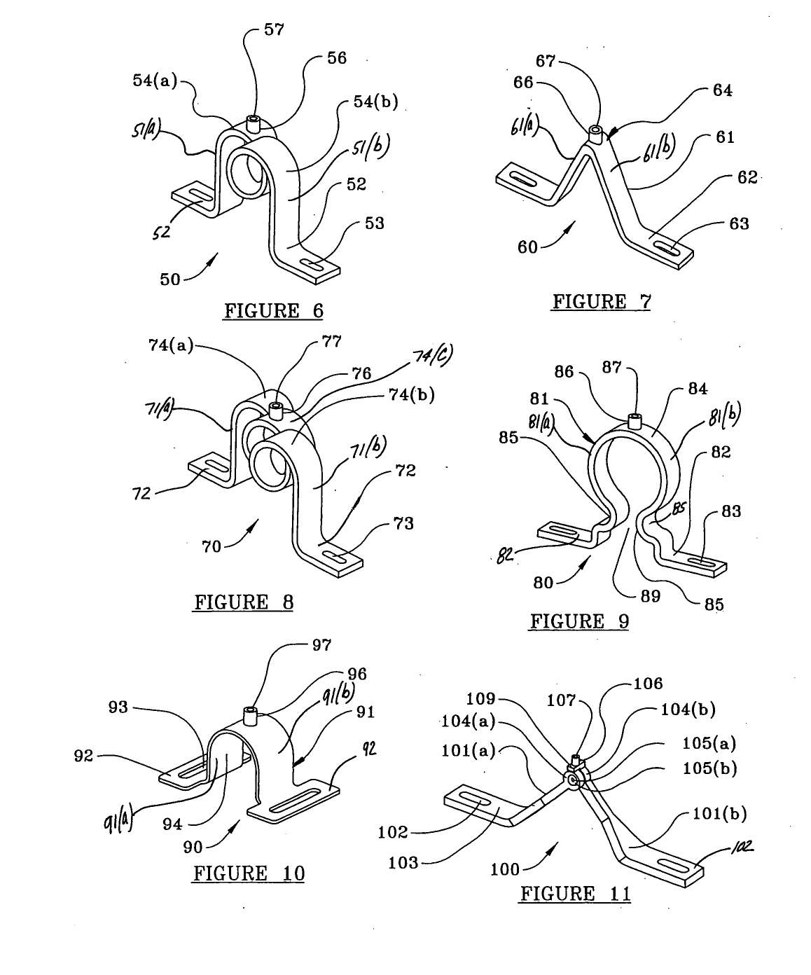

FIG. 6 shows another flexible bar embodiment 50 which is similar to the device shown in FIG. 5, except that it incorporates a pair of flexible, center looped portions 54(a) and (b) which are of uniform width and disposed in a side-by-side relationship. Feet 52 with slots 53 extend generally normally outwardly from the lower distal ends of the respective leg segments 51(a) and 51(b). Feet 52 are disposed in a parallel relationship to account for the displacement caused by center loop portions 54(a) and 54(b) being side by side. Cylinder/pin 56 with threaded bore 57 can be formed as a part of or attached to the top of either of the center portions 54(a) or (b), as shown.

FIG. 7 shows another flexible bar embodiment 60 in its unconstrained shape. In this embodiment, flexible center bridge portion 64 of body 61 is “V” shaped. Feet 62 with slots 63 extend outwardly from the lower distal ends of the respective leg segments 61(a) and 61(b) defined by “V” shaped center bridge portion 64. Bridge portion 64 of flexible bar 60 can also be shaped as a “W” or made in “WV” shape (not separately shown), or any similar configuration.

FIG. 8 shows yet another flexible bar embodiment 70 in its unconstrained shape. In this embodiment, flexible center portions 74(a), (b) and (c) define three loops, forming a coiled-spring shape. Leg segments 71(a) and 71(b) extend down from the ends of loops 74(a) and 74(b) respectively and the feet 72 (with slots 73) extend generally normally outwardly from the lower distal ends of leg segments 71(a) and 71(b) respectively. Cylinder/pin 76 with threaded bore 77 defined therein can be formed as a part of or attached to the top of center loop portion 74(c).

As seen in FIG. 9, still another flexible bar embodiment 80 consists of flexible bar body 81, whose center portion 84 is almost circular, and defines a pair of curved leg segments 81(a) and 81(b) with inwardly extending shoulders 85 formed along the lower respective ends of legs 81(a) and 81(b). Shoulders 85 are located between the feet 82 and the lower ends of legs 81(a) and 81(b). Each foot has a slot 83 through which a fastener such as a screw may be inserted. Cylinder/pin 86 is attached to or made an integral part of the top of the center, circular portion 84 of rod body 81 defines threaded bore 87.

Shoulders 85 form a space or opening 89 of a desired dimension between the feet 82. If shoulders 85 are brought inwardly towards each other into contact by angular movement of the vertebra to which feet 82 of bar 80 are attached, the degree of angular deflection of one vertebra with respect to the other is limited.

FIG. 10 illustrates a flexible plate/bracket embodiment 90 for attachment to cervical vertebra in its unconstrained shape. Because cervical vertebra are smaller than thoracic or lumbar vertebra, and because the device is typically inserted from an anterior or anterolateral approach, only a single flexible plate 90 is usually used. In this particular embodiment, the flexible center portion or member 94 of body 91 is wider and thinner than the corresponding center portion of earlier embodiments, is formed into a generally “V” shape and defines a pair of leg segments 91(a) and 91(b) terminating in feet 92 which contain slots 93, through which one or more screws may be inserted. Alternatively, center portion 94 of bar body 91 can be narrower and thicker to maintain desired levels of strength, durability and flexibility. Pin 96 with bore 97 protrudes outwardly from the top face of member 94. In this embodiment, feet 92 are wider than the leg segments 91(a) and 91(b) and the slots extend in a direction generally normal to the direction of the slots in the earlier embodiment.

Cylinder/pin 96, which contains threaded bore 97, may be attached to the center of the top face of center portion 94 of rod body 91 or made as an integral part thereof. The center portion 94 of plate 90 can be smaller in width than feet 92, the same width or wider.

FIG. 11 illustrates a hinged, moveable artificial spinal joint. In this preferred embodiment, rod or plate 100 has leg segments 101(a) and 101(b), which may be made of the same flexible or rigid materials as described above. The upper distal ends of segments 101(a) and (b) terminate in circular discs 104(a) and 104(b), respectively, each with a centrally located opening (not separately shown). Pin or rivet shaft 105(a) extends through the central opening (not separately shown) in discs 104(a) and 104(b). Thus, the leg segments are moveable and pivotable either towards or away from each other about the pin 105(a). Caps 105(b) are welded or otherwise attached, as known in the art, to the ends of pin or rivet 105(a) to prevent it from moving out of the central, circular opening in discs 104(a) and (b). The proximal ends of segments 101(a) and 101(b) terminate in flattened feet 102 with slots 103 for screws. Legs 101(a) and (b) are narrower and thicker than shown heretofore. This expedient can also apply to any of the other devices of FIGS. 2-10, 12, 13, 19 and 20. Legs 101(a) and (b) can also be made wider and thinner, if desired, maintaining their strength and durability.

Flange/bracket 109 may be formed as a part of or attached to the distal end of disc 104(b), as shown, and extends over the top of the distal end of disc 104(a). Alternatively, flange 109 can be attached to or formed as a part of disc 104(b) and extends over disc 104(a). In this embodiment, cylinder/pin 106, defining threaded, central bore 107, can be attached to or made a part of the top face of flange 109, as aforesaid. Alternatively, pin 106 may be made in flange 109 over the junction of discs 104(a) and (b), which may be widened or thickened to maintain its strength and durability.

FIG. 12 illustrates another moveable, artificial spinal joint device 110 consisting of leg segments 111(a) and 111(b), which may be made of any of the flexible or rigid materials described herein. The upper distal end of leg segment 111(a) is widened and defines a fork including two spaced-apart flanges or fingers 114(a) which are curled to receive pin or rivet shaft 115. The upper distal end portion of leg segment 111(b) terminates in a curled flange or finger 104(b), which is curled to receive pin or rivet shaft 115 and adapted to be fitted between the other two flanges, thus forming a hinge 118 about which the legs 111(a) and 111(b) are mounted for pivoting movement towards or away from each other. Rivet or pin caps (not separately shown) may be attached to the ends of rivet or pin shaft 115 to keep it from moving out of hinge 118. Device 110 can be made with any other number of varyingly configured flanges.

The lower proximal ends of leg segments 111(a) and 111(b) terminate in elongate flattened feet 112 with slots 113. Cylinder/pin 116, with central, threaded bore 117, can be attached to or formed as a part of center flange 114(b) of rod body 111(b). Alternatively, threaded bore hole 117 may be made in the center of center flange 114(b), which may be widened or thickened to maintain its strength and durability.

Optional metal band 119 surrounds the segments 111(a) and 111(b) and limits the angular outward movement of segments 111(a) and 111(b) away from each other to a desired angle and, hence, the angular movement of the vertebra. Metal band 119 has limited upwardly movement along leg segments 11(a) and (b) due to stop 111(c), which protrudes outwardly from the outside face of each of the leg segments 111(a) and (b), thus limiting the degree of movement of one vertebra with respect to the other.

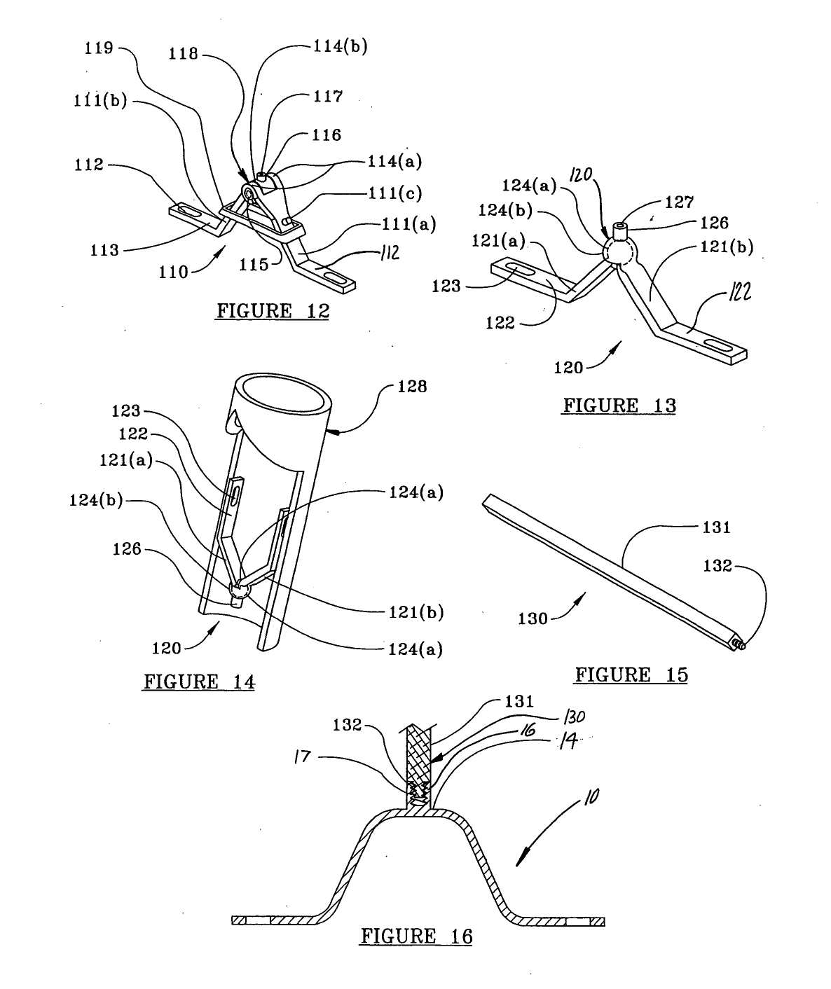

In a most preferred embodiment, as seen in FIG. 13, a moveable artificial spinal ball joint device 120 consists of leg segments 121(a) and 121(b), which may be made of any of the flexible or rigid materials described heretofore. The upper distal end of leg segment 121(a) terminates in ball 124(a), shown in phantom. The upper distal end of leg segment 121(b) terminates in cup or socket 124(b), whose interior is slightly larger in diameter than ball 124(a), at least 55% of which is moveably disposed within cup 124(b), forming ball joint 120. The lower proximal ends of segments 121(a) and 121(b) each terminate in elongate feet 122 with slots 123. Thus, leg segments 121(a) and 121(b) are moveable and pivotable towards and away from each other about ball joint 120.

As shown, a cylinder/pin 126 with central, threaded bore hole 127 can be attached to or formed as a part of the top face of the cup 124(b). Alternatively, threaded bore hole 127 may be made in the distal end of cup 124(b).

This embodiment has the benefit of movement similar to that of the facets. However, the movement of leg segment 121(a), which terminates in ball 124(a), is limited by the leading edge of the opening in cup 124(b), through which segment 121(a) extends, thereby limiting the degree of angular deflection of the vertebra.

FIG. 14 shows the device 120 of FIG. 13 disposed within delivery cannula 128. Any of the devices shown in FIGS. 2-12 can likewise be constrained or disposed within and delivered through a similar delivery cannula.

While moveable, artificial spinal joint devices 100, 110 and 120 of FIGS. 11-13 are depicted as “V” shaped, as seen in FIG. 7, moveable, artificial spinal joints 100, 110 and 120 may be made in any of the shapes shown in FIGS. 2-6 or 8-10 or any other desirable shape.

Any of the devices of FIGS. 2-13 can be alternatively made of a dual shape, superelastic memory metal NiTi alloy having appropriate and suitable proportions of nickel and titanium by weight. When cooled below its first shape's transition temperature the device assumes a shape enabling it to be inserted into the interior of the delivery cannula, without its having to be mechanically bent and constrained therewithin. After insertion into the body, when it reaches its second shape's transition temperature it assumes the unconstrained shapes shown in FIGS. 2-13.

Instead of being made of a flexible material for insertion through a delivery cannula as described above, any of the devices of FIGS. 1(a)-12(a) can be made of a rigid, non-flexible material, such as medical grade stainless steel, titanium, a titanium alloy, tantalum, carbon fibers in a matrix of a strong, durable and biocompatible plastic or other strong, durable and biocompatible material. Such devices can be inserted in conventional or moderately invasive surgical procedures, provided the artificial joint selected will fit within a delivery cannula with an inside diameter up to about 25 mm without having to bend its feet, enabling it to pass through the delivery cannula and be attached to the vertebra with screws, as known in the art, replacing the lattice of rods or plates and screws described heretofore.

As seen in FIG. 15, insertion tool 130, for insertion of a flexible or moveable artificial disc in a conventional, moderately invasive or minimally invasive surgical procedure, consists of a rectangular elongate tool arm 131 with a distally extending threaded projection or screw 132. While arm 131 of insertion tool 130 is shown as rectangular, arm 131 of insertion tool 130 can also be square, round or any other desired shape.

FIG. 16 illustrates threaded distal projection 132 of tool body 131 of insertion tool 130 of FIG. 15 threadingly engaged in threaded bore hole 17 in pin 16 of the device, which is an integral part of the center portion 14 of the flexible rod or bar device 10 shown in FIG. 2. Threaded distal projection 132 may, likewise, be threadingly engaged in the threaded bore hole of any of the devices of FIGS. 3-13 and 19-20.

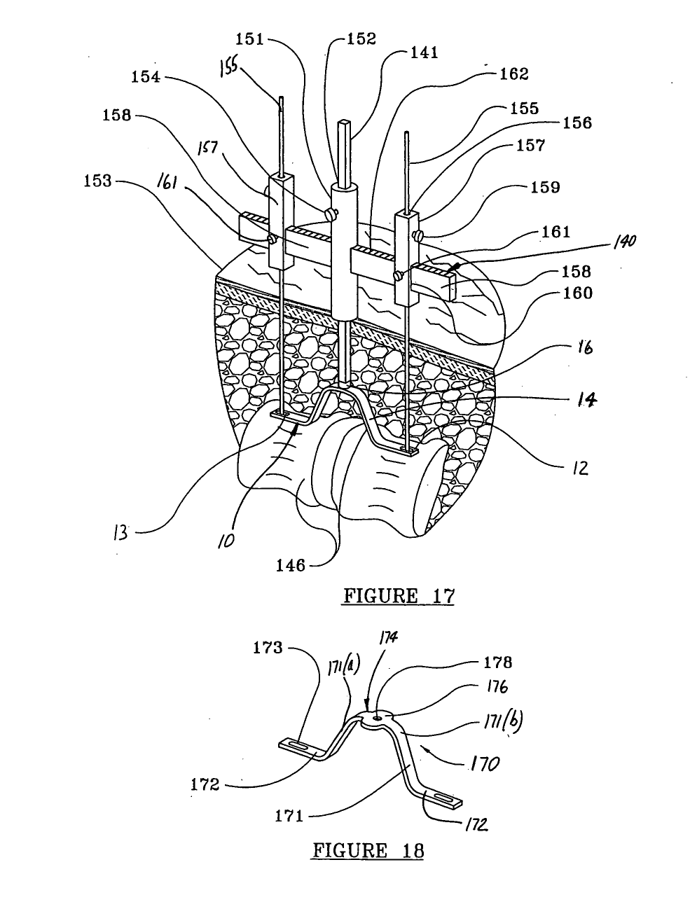

FIG. 17 illustrates a positioning jig 140, which is adapted to position screws for insertion through the slots 13 in the feet 12 of the flexible or moveable artificial joint device 10 into the vertebra, its facets or pedicles. Jig body 151, which contains a rectangular center hollow guide channel 152, is slid down, over rectangular insertion tool 141, whose threaded distal projection 142 has earlier been threadingly engaged in threaded bore hole 17 of metal cylinder 16 in the center portion 14 of the device 10 of FIG. 2, any of the devices of FIGS. 3-13 or 19-20 or a device of any other shape which has earlier been delivered up to vertebra 146, until the distal end of jig body 151 reaches the skin 153 of the patient.

The exterior of jig body 151 may alternatively be square, round or any other shape. The internal channel 152 of jig body 151 is the same shape and is slightly larger in size than the exterior of insertion tool body 141. Jig body 151 may be removably affixed to insertion tool body 141 by threaded set screw 154, as known in the art.

Jig 140 additionally includes a pair of side guideway members 157 which are operably coupled to center guide 152 by means of a horizontal support bar 158. Members 157 are generally vertically oriented and spaced from and parallel to guide 152. Each of the members 157 defines a hollow interior guide channel 156 adapted to receive guidewire or trocar 155 which terminates in a sharp point or trocar shape (not separately shown). Slots 160 in the hollow guideway member 157 allow the hollow guideway members 157 to be slid over the ends of the arms of bar 158. The surgeon, knowing the positions at which the feet of the artificial joint are to be attached to the vertebra facets or pedicles, selects an appropriately sized artificial joint, the dimensions of whose slots are known. Guideway members 157 are slid along bar 158 and then removeably secured to bar 158 by respective tightening set screws 161. Markings 162 located along the top face of the bar 159 enable the surgeon to fix hollow guideways in place at the proper position.

The sharp distal ends of guidewires or trocars 155 are adapted to be penetrated through the patient's back, passed through slots 13 of feet 12 of flexible rod or plate device 10 of FIG. 2 (or those of any of the other devices) and docked in the vertebra, as shown, or in the pedicles or facets of the vertebra (not separately shown).

While the channel 156 in each of the hollow guideway members 157 and guidewire or trocar 155 may both be round, both may be square, rectangular or any other desired, matching shapes. Jig 140 positions guidewires or trocars 155 so they may be inserted through slots 13 of feet 12 without the need to create a sizeable surgical incision.

Guidewires or trocars 155 can be approximately the same diameter as the screws to be inserted through slots 13 of feet 12. If desired, after insertion, guidewires or trocars 155 may be removed, tissue expanders, as known in the art, may be inserted through the passageways created by guidewires or trocars 155, to widen the passsageways for the insertion of screws (not separately shown). The screws used to attach rod or plate 10 of FIG. 1(a) may be made of medical grade stainless steel, titanium or other strong, durable and biocompatible material, and may be removably attached to a screwdriver, as known in the art.

The screws, removably attached to screwdrivers, may be inserted through channels 156 in hollow guideways 157, the passageways created by guidewires or trocars 156, or into widened passageways created by tissue expanders, and slots 13 of feet 12. They may then be screwed into place in the desired bony structures, the caps of the screws being wider than slots 13 in feet 12 of the artificial spinal joint.

While the flexible rod or plate shown in FIG. 17 is rounded, as seen in FIG. 2, the rod or plate or a moveable artificial spinal joint can be made in any of the shapes shown in FIGS. 2-13 or of any other desired shape, of a flexible or rigid, durable material, or of a dual shape, superelastic memory metal, as described above.

Instead of the exterior of insertion tool 141 being rectangular, square or hexagonal, and central channel 152 in body 151 having the same, but slightly larger shape, for example, insertion tool 141 can be round with an external ridge or key along its length (not shown), and the central channel of jig body 151 can have an internal, round channel 152, which is slightly larger in diameter than the outside diameter of insertion tool 141, with an indentation or keyway along its exterior (not shown), into which the external ridge or key of insertion tool 141 can be moveably inserted. Alternatively, insertion tool 141 can have the indentation or keyway and jig body 151 can have the external ridge or key. Any other registration means known in the art can be employed to accomplish the same positioning purpose.

As shown in FIG. 18, instead of cylinders or pins with threaded bore holes, as seen in FIGS. 2-13 being attached to or formed as an integral part of the central portion of a rod, plate or moveable artificial joint, the rod or plate body 171 of device 170 may have a widened, as shown, or thickened center post portion 176 defined at the top of the generally “V” shaped center portion 174, in which threaded bore hole 178 can be made, maintaining the strength and durability of center portion 176 of device 170. Feet 172 with slots 173 extend outwardly from the lower end portions of the legs 171(a) and 171(b) of center portion 174. The center portion of the rod or plate body of any of the devices of FIGS. 2-13 ands 19-20 may, likewise incorporate a similar post.

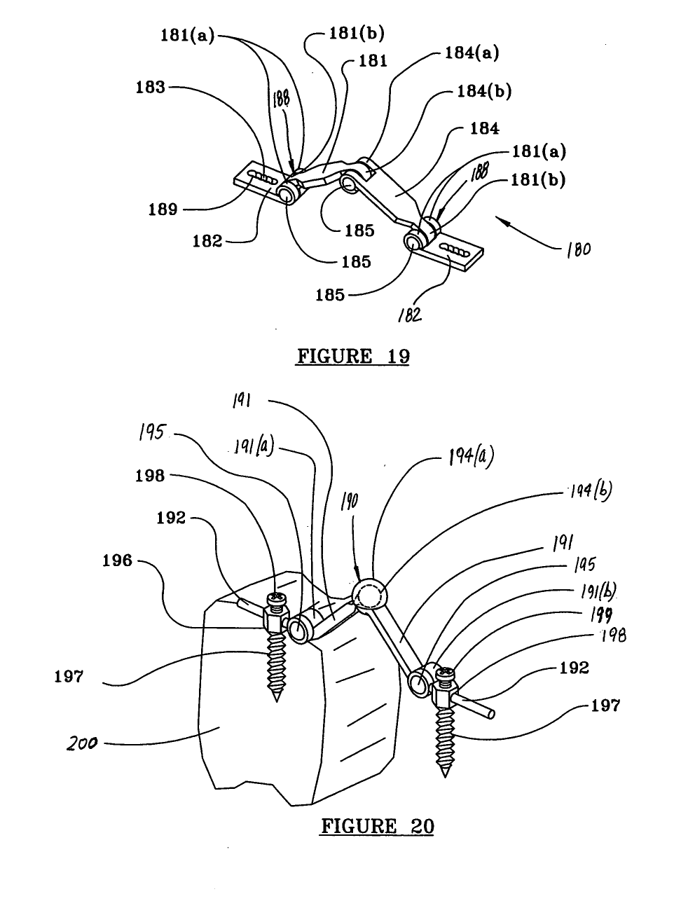

In FIG. 19, a most preferred embodiment of the device of the present invention is shown. Artificial spinal joint device 180 may be made of any of the flexible or rigid materials described above, and may be made in any of the shapes shown in FIGS. 2-13 or any other desirable shape. However, in this embodiment, the feet 182 are moveably joined to the lower ends of the flexible or rigid rod or plate leg segments 181 and 184 by a pin or rivet, as described in FIG. 11, by a hinge, as described in FIG. 12 or by a ball joint, as described in FIG. 13, or by any other moveable joining means known in the art. The benefit of this construction is that the feet of the artificial spinal joint need not be manually bent or thermally caused to assume a shape that permits the artificial spinal joint to be inserted through a delivery cannula with an interior diameter of about 14 mm or less.

As shown, the proximal end of feet 182 are wider than as shown in FIGS. 2-13, and each of which is formed into two flanges 181(a) defining a fork and which are spaced apart and curled about pin or rivet shaft 185. The lower proximal end of rod or plate segment 181 terminates in single flange 181(b), which is disposed in the space between flanges 181(a) and is likewise curled about pin or rivet shaft 185, forming hinge 188. Caps or flanges (not shown) may be formed as a part of or attached to the ends of pin or rivet shaft 185 to prevent shaft 185 from moving out of flanges 181(a) and (b).

This embodiment enables feet 182 of artificial spinal joint 180 to be folded or pivoted about the lower ends of leg segments 181 and 184 for insertion through a delivery cannula in a minimally invasive procedure, without having to excessively compress the device, or through an incision in a moderately invasive or conventional surgical procedure. During insertion, feet 182 are positioned parallel to the axis of rod or plate leg segments 181 and 184. Prior to attaching feet 182 to the vertebra, pedicles or facets, feet 182 are moved outward to an angle of about 60° to 90° from the axis of rod or plate segments 181 and 184.

In this embodiment, the interior face edges 189 of slots 183 in feet 182 are scalloped, preferably with rows of about one-fourth to one-half circles, each aperture (partial circle) being wide enough to permit a screw to pass through. However, the points of the scallops (ends of the partial circles) are of a size, which do not permit the screw to move out of its aperture. The benefit of this construction is, if a screw becomes loose over time, the foot's position on the vertebra is better maintained than if the slots were unconstrictive. Optionally, the feet of all of the devices of FIGS. 2-13 may have scalloped slots, such as seen in FIG. 19.

Alternatively, as seen in FIG. 20, which shows another device embodiment 190 which is of the ball/socket type shown in FIG. 13 and thus incorporates a pair of legs 191 terminating respectively at one end into a ball socket 194(a) and ball 194(b) and terminating at the other ends thereof into respective curled flange ends 191(a) and 191(b) adapted to receive respective pins 195 therethrough. Device 190 additionally includes a pair of elongate cylindrical shafts 192 adapted for pivotal movement about the pins 195. A hexagonal nut 196 defines a horizontal port or through aperture 198 which allows the nut 196 to be slid for movement along the length of each of the shafts 192. A vertical through aperture in each of the nuts 196 allows a set screw 199 to be secured therein for fixing the position of nut 196 along the length of shafts 192. A screw 197 extends out of the lower face of each of the nuts 196. Screws 197 are adapted to be fastened as earlier described into vertebra 198 (or a pedicle or facet thereof) by a tool (not shown) turning hexagonal nut 196 clockwise, as known in the art. This construction allows artificial joint 180 to be positioned for attachment to the pedicles, facets or vertebra 198 and then fastened in place. Optionally, nuts 196 may be square, triangular, hexagonal or any other feasible shape.

Any combination of the features described above may be used in the construction of a flexible or moveable artificial joint. For example, the center portion can be flexible, rigid or moveable, and the feet may be flexible or rigid and moveably attached thereto.

The flexible artificial spinal joints or moveable artificial spinal joints described above may be employed to replace a non-functional or stenosed spinal joint (facet), for example, in persons to correct a spinal deformity such as scoliosis or spondylolisthesis, to replace a spinal joint which is painful, damaged, diseased or otherwise not functional, or to replace a spinal joint which is non-functional in a person whose disc is degenerated and a spacer is inserted, as well as in the treatment of other conditions where an artificial spinal joint is desirable.

Flexible artificial spinal joints or flexible, moveable artificial spinal joints made of any of the flexible materials described above may be compressed, delivered and attached to the desired bony structures of the vertebra, preventing subluxation and retaining the mobility of the spine by providing for angular deflection of one vertebra with respect to the other, in minimally or moderately invasive procedures, or in conventional surgical procedures, with or without a delivery cannula. Rigid embodiments of the moveable artificial spinal joints described herein may be inserted in conventional surgical procedures or, with feet moveably attached to the rod or plate segments of the moveable artificial spinal joints, in moderately or minimally invasive procedures.

While this invention is susceptible of embodiment in many different forms, there are shown in the drawings and will be described in detail herein specific embodiments thereof, with the understanding that the present disclosure is to be considered as an exemplification of the principles of the invention and is not to be limited to the specific embodiments illustrated.

Numerous variations and modifications of the embodiments described above can be effected without departing from the spirit and scope of the novel features of the invention. It is to be understood that no limitation with respect to the specific apparatus illustrated herein is intended or should be inferred. It is, of course, intended to cover by the appended claims, all such modifications as fall within the scope of the claims.

1. An artificial spinal joint, comprising at least one member whose center portion is flexible and whose distal ends terminate in feet that contain slots through which at least one fastener may be inserted to attach the feet to vertebra whose facets are non-functional.

2. The artificial spinal joint of claim 1, wherein the member is made of a flexible material selected from a group comprised of spring steel coated with a durable, bio-compatible material, a strong, flexible, durable plastic, carbon fibers in a matrix of a strong, flexible, durable plastic, a single shape, superelastic memory metal consisting of nickel and titanium, whose shape is fixed by heat treatment at a specific temperature, and a dual shape, superelastic memory metal, consisting of nickel and titanium which, after heat treatment, has a first shape below a selected temperature and a second shape at a temperature at least equal to the selected temperature.

3. The artificial spinal joint of claim 1, wherein the member is made of a rigid material selected from a group consisting of medical grade stainless steel, titanium, a titanium alloy, tantalum, and carbon fibers in a matrix of a rigid, durable plastic.

4. The artificial spinal joint of claim 1, wherein the center portion of the member is bent into “C”, “V”, “head and shoulders”, “W”, loop or coiled shape.

5. The artificial spinal joint of claim 1, wherein the inner surface of the slots is scalloped, with the distance between the points of the opposing scallops being smaller than the diameter of the screws to be used to attach the artificial spinal joint to the vertebra.

6. A moveable, artificial spinal joint consisting of at least two segments, the distal ends of which are moveably joined together and the proximal ends of which terminate in feet which have slots through which at least one screw may be inserted to attach the feet to vertebra whose facets are non-functional.

7. The artificial spinal joint of claim 6, wherein the segments and feet are each made of a material selected from a group comprised of titanium, a titanium alloy, tantalum, medical grade stainless steel, spring steel coated with a durable, bio-compatible material, a bio-compatible plastic material, carbon fibers in a matrix of a durable plastic material, a single shape superelastic memory metal of nickel and titanium whose shape is fixed by heat treatment, and a dual shape superelastic memory metal of nickel and titanium which after heat treatment has a first shape below a selected temperature and a second shape at a temperature at least equal to the selected temperature.

8. The artificial spinal joint of claim 6, wherein the distal ends of the segments are disc-shaped, define a channel and are moveably joined together with a pin extending through said channel.

9. The artificial spinal joint of claim 6, wherein the distal ends of the segments define flanges forming a hinge about a pin.

10. The artificial spinal joint of claim 6, wherein the distal end of one segment is a ball, and the distal end of the other segment is a ball socket, within which at least 60% of the said ball is moveably disposed.

11. A moveable, artificial spinal joint having at least two segments, the distal ends of which are moveably joined together, and the proximal ends of which are moveably attached to feet with slots through which at least one screw may be inserted for attachment of the feet to vertebra whose facets are non-functional.

12. The artificial spinal joint of claim 11, wherein the inner surface of the slots is scalloped, with the distance between points of the scallops being smaller than the diameter of the screws to be used to attach the artificial spinal joint to the vertebra.

13. A method for inserting and attaching an artificial spinal joint to vertebra whose facets are non-functional, including the steps of:
(a) creating a passageway up to the vertebra for insertion of a delivery cannula;
(b) removably attaching an insertion tool to the center of an artificial spinal joint;
(c) compressing the artificial spinal joint and inserting it into the delivery cannula;
(d) retracting the delivery cannula at least a sufficient distance to allow the artificial spinal joint to resume its unconstrained shape;
(e) removably inserting a jig over the insertion tool;
(f) inserting sharply pointed guides through channels in the said hollow guideways, through tissue and into the surface of the bony structure of the vertebra to which the feet of the artificial spinal joint are to be attached;
(g) removing the guides and inserting screws removably attached to a screwdriver through the guideways, the tissue and the slots in the feet of the artificial spinal joint; and
(h) securing the screws into the desired bony structures of the vertebra.

14. An artificial spinal joint comprising a pair of legs terminating in a pair of feet at the respective ends of the legs which feet are flexible about the legs.

15. The artificial spinal joint of claim 14 wherein the feet and legs are made of a shape memory alloy which allows the feet to flex relative to the legs.

16. The artificial spinal joint of claim 14 wherein the feet are pivotable about the ends of the legs.

17. The artificial spinal joint of claim 14 wherein the terminal end portions of the legs opposite the feet are pivotally joined together to allow the movement of said legs towards or away from each other.

18. The artificial spinal joint of claim 14 wherein the ends of the legs opposite the feet are unitary with a spring member for flexing said legs towards or away form each other.

19. The artificial spinal joint of claim 14 wherein the ends of the legs opposite the feet cooperate together to define a spring for flexing said legs towards or away from each other.

20. The artificial spinal joint of claim 14 comprising an elongate bar which has been bent and shaped so as to define the legs and feet.
